(19) United States
(12) Patent Application Publication
(10) Pub. No.: US 2010/0324627 A1
(43) Pub. Date: Dec. 23, 2010

(54) METHOD AND APPARATUS FOR RESISTIVITY MEASUREMENT, DETECTION AND TREATMENT IN LIVING TISSUE

(75) Inventor: Ronald J. Weinstock, Newport Coast, CA (US)

Correspondence Address: NIXON PEABODY, LLP, 300 S. Riverside Plaza, 16th Floor, CHICAGO, IL, 60606-6613, US

(73) Assignee: NewLife Sciences, LLC, Beverly Hills, CA (US)

(21) Appl. No.: 12/709,088

(22) Filed: Feb. 19, 2010

Related U.S. Application Data
(63) Continuation of application No. 12/460,999, filed on Jul. 28, 2009.
(60) Provisional application No. 61/084,187, filed on Jul. 28, 2008.

(51) Int. Cl. A61N 1/36

(52) U.S. Cl. 607/62

(57) ABSTRACT

An electrotherapeutic system including a device and method provides beneficial reduction of pain, bruising, swelling and other maladies. The electrotherapy system uses a measuring current to identify precise locations preferred for application of electrotherapy. A treatment current is applied at the points identified. Use of probes capable of applying both measuring and treating currents are used with an audio signal to locate and treat afflicted regions of a patient.

### CROSS-REFERENCE TO RELATED APPLICATION

This application is a continuation of U.S. patent application Ser. No. 12/460,999, filed Jul. 28, 2009, which claims the benefit of priority to U.S. Provisional Application No. 61/084,187, filed on Jul. 28, 2008 entitled “Method and Apparatus for Resistivity Measurement, Detection and Treatment in Living Tissue,” each of which is hereby incorporated by reference herein in its entirety.

### COPYRIGHT

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND OF THE INVENTION

1. Field of the Invention

The present invention relates to a system and method of treatment for living tissue using electric current. More particularly, the invention relates to a system for identifying the optimal point for applying electrotherapeutic treatment and the optimal type of electric current for affecting a quantifiable effect on a patient.

2. Prior Art

Electrotherapy, or the application of electricity in one form or another as therapeutic treatment, has been widely explored in the medical profession for a broad range of uses. Electricity has been applied to people with medical problems for centuries in the hope of promoting healing, treating incurable diseases and psychological disorders, for both growing and removing hair, and a nimiety of other purposes. Success has typically been sporadic and unpredictable.

Broad success has been found in the application of electricity for muscle and nerve stimulation. Techniques have been successfully developed to stimulate muscle contraction in order to help train specific muscles, perform physical therapy and even nerve replacement in people suffering from nerve damage. Neuroelectrostimulation is increasingly finding applications as the nervous system becomes better understood. Electrotherapy is carving out a role for itself in a variety of disciplines including acupuncture, kinesiology, and stress management. However, attempts to elicit analgesic, healing and other effects from electrotherapy have been elusive despite prolific effort on the part of medical profession.

U.S. Pat. No. 5,374,283 to Flick discloses an electrical therapeutic apparatus used for the treatment of pain. It consists of two electrodes attached to broad pieces of fabric coated with conductive material which are applicable to different parts of the human body. The apparatus generates a uniform current, either AC or DC which envelops the portion of body inside the fabric. It does not disclose a method or apparatus used for determining baseline resistance or finding areas of high resistance in living tissue, nor does it disclose a method of increasing current to treat these areas of high resistance. It also does not disclose an audio or visual cue system for use by the practitioner during treatment. It further does not disclose a method of applying localized current, but rather, distributes current over a broad surface.

U.S. Pat. No. 5,573,552 to Hansjurgens discloses an apparatus and method for applying electric current to a body part to treat pain associated with the area. The apparatus consists of a circuit that generates a medium-frequency between 1000 Hz and 100,000 Hz. It also has two electrodes that receive the output and a frequency modulator attached to the circuit. It does not disclose the use of a circuit that generates frequencies of 400 Hz to lessen resistivity, in fact, it suggests that frequencies in that range are likely to increase rather than decrease pain. It also does not suggest the use of a pulsed current, and in fact suggests that a pulsed current increases resistance rather than decreases resistance.

U.S. Pat. No. 6,014,585 to Stoddard discloses a method of treating pain through the use of electrical or ion conducting tape. The tape consists of different layers of adhesive material, conductive material, and protective material. It is pressed directly onto the area of pain with no external electric current utilized with the goal of restoring electrical connections between cells. It does not disclose a method of applying external current to precise trigger points through the use of external probes and a series of switches. It further does not disclose audio or visual cues for locating and treating areas of high resistance in living tissue. It further does not disclose a method of applying increased electric current to areas of high resistance with the goal of removing the areas of high resistance. It merely discloses a method of applying sustained current to a broad area of tissue, entirely bypassing the areas of resistance.

U.S. Pat. No. 6,085,115 to Weaver et al. discloses a method for measuring biopotential of an organism through the electroporation of tissue. It discloses a method for decreasing resistance for a short period of time and in fact involves a potential increase in pain. It does not disclose a method of decreasing pain by decreasing resistance. It also does not disclose an apparatus which searches for higher levels of resistance across tissue through the use of external probes and a series of switches. Nor does it disclose a method of reducing resistance with a goal of reducing pain, in fact, it discloses a method of reducing resistance that in turn has the potential to cause pain.

U.S. Pat. No. 6,546,290 B1 to Shloznikov discloses a method and apparatus for providing therapeutic electrical signals in a predetermined pattern and constructed in a manner so that a user can use the apparatus herself. It provides a plurality of electrodes so that a large area of the body may be treated at one time. It also provides a platform that conforms to the shape of the area to which the therapy is applied. It does not disclose a method of finding areas of greater resistance, nor does it disclose a method of auditory and visual signals alerting the practitioner to the resistivity of a given area of tissue. It further does not disclose the use of external probes to scan an area of tissue for resistivity.

U.S. Pat. No. 7,117,034 B2 to Kronberg discloses an apparatus and method for generating an electrical signal for use in biomedical applications. It has an adjustable output signal generated by blending two timing intervals to generate a variable frequency current, preferably with no net DC current. It does not disclose a method of reducing resistance across a given area by applying pulsed current.

U.S. Pat. No. 7,353,058 B2 to Weng et al. discloses a device which detects and measures impedance of a pressure point. It has a one button hand-held setup with an LED and beeping noise to signal completion of the measurement. It suggests that a region of low impedance is best suited for application of electric current. It does not suggest or disclose a method for measuring an area of high impedance and method of removing the impedance through electric pulses.

International Patent Application, Publication No. WO 2007/138595 A2 to Naroditsky discloses an apparatus and method which identifies acupuncture points for use with TENS applications. A device is applied to a region of the patient's skin and the area of lowest impedance is identified as the preferred spot for applying electric current. It does not disclose an apparatus or method which identifies points of the highest resistance, in fact, it does directly the opposite.

Despite the accomplishments described above and sundry other efforts, effective electrotherapy has remained elusive. Means to properly and accurately identify the precise location at which to apply current and the optimal type of current vary widely depending upon which electrotherapy theories are applied. Although it has been generally shown that areas afflicted with pain or injury typically exhibit lower conductivity, i.e. higher resistivity, than healthy areas of the body, there have been no know effective applications of this phenomenon. Many methods of applying electricity to living tissue have been developed, including a nimiety of specially designed electrodes. However none have proven particularly effective in properly applying electricity to effectively treat tissue in a manner that may be quantified. The types and strength of electric current applied vary widely depending on the theories used to account for any electrotherapeutic effect.

It is therefore desirable to provide a method for accurately identifying a precise location to which an electrotherapeutic current should be applied.

It is also desirable to provide an effective type of current having optimal electrotherapeutic qualities.

It is also desirable to provide a means to quantify the therapeutic effect of electrotherapy.

### BRIEF SUMMARY OF THE INVENTION

The present invention provides a means for identifying the precise region to which electrotherapy may be applied to facilitate maximum effectiveness, optimal application of electrotherapy to the identified region and means for quantifying the therapeutic effect of the treatment provided by the invention. The invention synergistically combines a method of identifying the optimal region for applying electrotherapy and an optimal method for applying electrotherapy to the identified region. The invention also preferably utilizes an electrotherapeutic apparatus that maximizes the efficiency, accuracy and synergy between the diagnosis and treatment of afflicted tissue.

An electrotherapist uses two electrodes that are moved about an afflicted region of a patient. The conductivity of the patient's body is measured as the electrodes are moved until a region of relatively high resistivity is identified. A pulsed direct current is applied to the two points at which the electrodes encounter high resistivity. After brief application of the pulsed current, the resistivity is again measured. A pulsed current is applied to the selected region until the resistivity of the region drops close to a baseline level of conductance. Generally, the reduction of resistivity coincides with alleviation of pain, bruising or other discomforting affliction.

Preferably a device having two probes is used for the treatment process of the invention. The probes include electrodes that may be used to apply a low power current to measure conductance/resistivity and may also be used to apply stronger, pulsed current to treat a patient. One of the probes preferably includes controls to switch between the two types of applied currents. The power source to which the electrode probes are attached includes at least one output that quantifies the level of resistance. This allows the amount of resistance in the region being treated to be measured so that the efficacy of the treatment may be determined. Preferably the output for resistance measurement includes an audio output so that an electrotherapist can identify a region of high resistance by listening so that his or her eyes may remain on the positioning of the electrode probes during treatment.

It is often desirable when applying the invention to first identify a baseline level of resistance by measuring conductance across an unafflicted portion of a patient's body.

It is therefore an object of the present invention to provide a system for measuring conductance and resistance over a portion of the body of a patient.

It is another object of the invention to provide a system for applying an electrotherapeutic current to a patient.

It is another object of the invention to provide a system for quantifying the efficacy of an electrotherapeutic treatment method.

The above broadly outlines the more important features of the invention in order that the detailed description thereof that follows may be better understood, and in order that the present contribution to the art may be better appreciated. The features of the invention are more thoroughly described hereinafter and will form the subject matter of the claims.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting unless such an intention is explicitly indicated herein.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

### BRIEF DESCRIPTION OF THE DRAWINGS

FIG. 1 is an environmental view of the invention.

FIG. 2 is a perspective view of an apparatus of the invention.

FIG. 3 is a flow chart of the invention.

FIG. 4 is a chart showing a typical measuring current of the invention for measuring resistance.

FIG. 5 is a diagram of a square wave useable as a therapeutic treating current for the invention.

FIG. 6 is a chart showing a typical electrotherapeutic treating current of the invention.

FIG. 7 is a schematic diagram of an embodiment of the device of the invention.

### DETAILED DESCRIPTION OF THE INVENTION

The present invention is designed primarily for the treatment of living tissue, specifically human patients experiencing pain, bruising, arthritis and other forms of discomfort or maladies. A pair of probes having electrodes are moved across the afflicted area to be treated and transmit a small current. Preferably hydrogel is used to facilitate current flow into a patient's tissue. The probes are connected to an electrotherapy device having a display showing the amount of resistance between the two probes. Preferably, the electrotherapy device also includes a speaker that emits noise at a frequency that corresponds to the amount of resistance between the two probes, such that the lower the electrical resistance encountered by the current emitted by the probes, the higher the emitted frequency. This allows the operator to identify the placement points of the probes that results in the highest amount of resistance without the distraction resulting from a need for the operator to look at a visual readout.

Once the operator has identified the placement of the probes that results in the highest resistance, the probes are switched to a treatment mode in which they emit an electrotherapeutic current. This electrotherapeutic current is applied for several seconds. The probes are then switched back to the analyzing mode. The application of the therapeutic current decreases the resistance of the region being treated. This reduction in resistance is observed and if resistance is sufficiently reduced, then treatment will end. If there is still substantial resistivity or if there is still substantial pain, treatment with the electrotherapeutic current may continue and subsequent measurements may be made. The process may be repeated as many times as desired, but is typically discontinued once the resistance between the probes falls to the baseline level.

To determine a baseline level of resistivity, the probes may be applied to an area of the patient unaffected by pain, bruising or other maladies. It is preferable to reset a baseline for the instrument being used for each individual patient. Each individual has unique bioelectrical properties that fluctuate. Any random area of the patient, preferably away from the carotid cavity, the brain, and other areas of substantial bioelectric activity, may be used to establish a baseline.

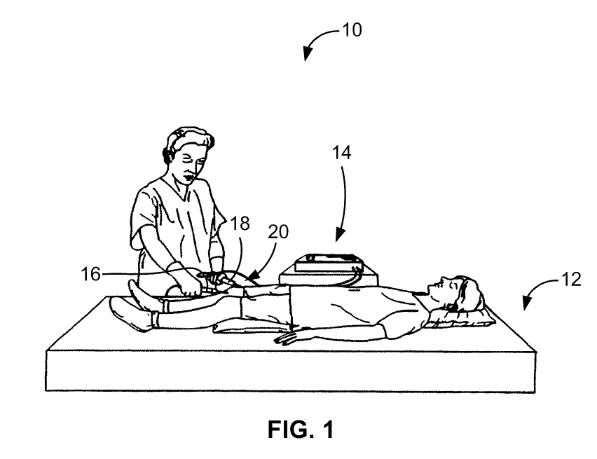

FIG. 1 shows an environmental of view of the system and method of the apparatus in use for the treatment of a patient. Operator 10 is using electrotherapy device 14 to alleviate pain and swelling in afflicted region 20 of patient 12. The patient 12 is preferably placed in a reclined position in which his or her muscle and other tissues are relaxed. Primary probe 16 and secondary probe 18 are held by the operator 10 and connected to electrotherapy device 14. Electrotherapy device 14 supplies power to the probes 16 and 18 and includes instrumentation for measuring current flow between the probes, controlling the type and power of current supplied to the probes, and indicates the conductivity between the probes preferably using both visual and audio signals.

In the view shown in FIG. 1, the operator 10 first applies a conductive hydrogel to the afflicted area 20. Probes 16 and 18 are then placed in contact with different points on the afflicted area 20. Electrotherapy device 14 emits a sound, the volume of which correlates with the amount of resistance across the current pathway over the afflicted area. As the operator 10 moves the probes 16 and 18 about the afflicted region 20, the current pathway changes, leading to a change in the resistivity and therefore the volume of the emitted tone increases or decreases accordingly. The use of an audio signal allows the operator 10 to hold a constant gaze at the afflicted region 20 and the positioning of the probes 16 and 18. Were the output of the electrotherapy device visual only, the operator would be required to continuously shift his or her gaze between the visual readout display and the afflicted region. Use of an audio signal, therefore, increases the speed and accuracy of treatment.

The probes 16 and 18 are moved about the afflicted region 20 until the positioning that provides the highest resistance to the current is located. The operator then uses the switch, preferably located on the primary probe, to switch the current supplied to the probes by the electrotherapy device from a diagnostic, measuring current to a treatment current. The treatment current is a pulsed direct current having substantially higher voltage than that of the constant and steady measuring current. The treatment current is applied for several seconds, usually from about 20 seconds to about a minute. The operator then toggles the switch again to revert the device back to a measuring current. Typically, after application of a treatment current, the resistance to the current between the two probes over the afflicted region has decreased. If the resistance has decreased sufficiently such that it is close to the baseline level of resistance, then treatment is typically discontinued. However, if resistance is still relatively high in the region, the operator may choose to reapply the treatment current. Resistance is then measured again. This process is repeated until the resistance has decreased sufficiently. The operator may then move the probes about the region, looking for other positionings of the probes that results in high resistance to the measuring current.

In the manner described above, an operator may quickly and accurately apply electrotherapy to a patient for treating a variety of afflictions, including, but not limited to reduction of acute or chronic pain, post-surgery bruising and swelling, hematomas, tendonitis, osteoarthritis, and a host of other maladies. It is also effective in stimulating bone growth, enhancing tissue regeneration, promoting wound healing, and increasing mobility of patients with multiple sclerosis. The electrotherapy of the present invention is also effective in reducing tremors resulting from Parkinson's disease, delirium tremens, and other ailments. The exact mechanism of treatment is unknown, although theories abound. The efficacy of the invention is promoted by the accuracy with which it pinpoints the precise location and probe placement, as determined by measuring resistivity/conductivity of the tissue, to which a pulsed direct current may be applied for maximum benefit to the patient.

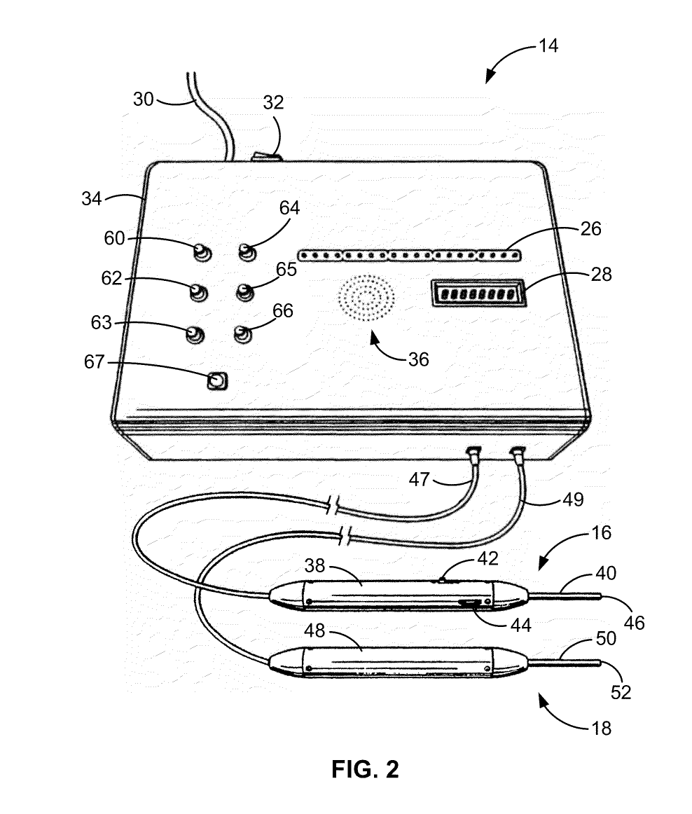

FIG. 2 shows a preferred embodiment of the electrotherapy device 14. The electrotherapy device is relatively simple electronically yet provides a very effective system for accurately treating a variety of afflictions using electrotherapy. Casing 34 hold the circuitry of device 14, which is supplied power from a typical outlet by means of power cord 30. However, as the invention is explained below in more detail, a skilled artisan will appreciate that a variety of power sources are readily adaptable for use with the invention.

The circuitry of device 14 held within casing 34 is not shown but is readily available. It is capable of providing two types of current. The measuring current is a steady direct current, preferably about 12 volts. This current is relatively steady and may be of any voltage that does not cause any significant discomfort to a patient when applied. The other current, the treatment current, is a pulsed direct current. The pulsed current is comprised of bursts of direct current that form a typical square wave of about 50 volts. This treatment current is substantially stronger than the measuring current. The circuitry of the device allows both types of currents to be fed to the electrodes in the probes of the invention. The circuitry includes current measuring devices for determining the conductance and resistance of the electrical circuit created between the electrodes in the probes by the organic tissue contacted by the probes. The circuitry also includes one or more indicators as described below to signal the amount of resistance by means of a digital readout, a light bar, an audio signal, combinations of all three or the like.

A master on/off switch 32 is preferably provided and preferably includes a surge protection means. Casing 34 of device 14 also preferably includes a speaker 36 for emitting sounds that correlate to the level of current between probes 16 and 18 during the measuring and diagnostic functions of the device. Display 28 is a digital readout. Display 28 may be used to display a variety of different information, including a quantified value for the level of resistance or conductance within the current during diagnosis, total time of exposure of the patient to a particular type of current, power level of the device and other data that may be of interest as appreciated by those skilled in the art. The value may be picked arbitrarily or may be an actual value as measured in ohms of resistance or amperes of current. The digital readout may also be used in conjunction with an Algorithmic Evaluation Code (AEC). The AEC may be a numerical value calculated from one or more parameters using programming comprised of hardware, software, and/or firmware and developed to serve as an indicator of resistance and/or conductivity of organic tissue. Because the purpose of measuring this value is primarily to establish a base line value and measure the deviation from this value, the actual numeric value of the number is often not relevant. Display 28 is also a preferable, but not essential feature of the invention. Those skilled in the art will appreciate that the digital readout may also be used to provide other data regarding the various functionalities of the machine.

Device 14 also includes a light readout 26 that is usually comprised of an LED display. The light readout is typically a long row of lights. The amount of resistance or conductivity is shown by the number of lights that are lit. The light readout 26, the display 28 and the speaker 36 are all alternative means by which the device 14 indicates the level of resistance or conductance of the current passing through the patient between the electrodes. The display 28 is beneficial because it provides a numerical value for comparison to other values. The audio signal from speaker 36 is beneficial because an operator can monitor the sound level and therefore resistance of the current without taking his or her eyes off of the region being treated. The light readout provides another method of indicating the level of resistance in the current. Which device or combination of devices is used may vary between different embodiments.

Primary probe 16 includes a probe body 38 that is preferably shaped to ergonomically fit easily in an operator's hand. It may optionally include a surface having a shape and/or texture to enhance steady and firm gripping. Primary probe 16 also includes electrode 40 that protrudes out of the front and is typically long and relatively thin, preferably having a rounded tip 46. The thin shape of electrode 40 allows for precise placement while the rounded tip 46 makes the electrode more comfortable for the patient and facilitates moving of the electrode about the area being treated. The primary probe is connected to the device case 34 by wire 47 which supplies current to electrode 40.

Primary probe 16 includes two controls by which the operator may control the device without removing his hands or even looking directly at primary probe 16. On the body 38 of primary probe 16 is a treatment switch 42 for alternating between the diagnostic measuring mode to the treatment mode of the device. The operator may use the finger or thumb of the hand holding the primary probe to toggle the treatment switch 42 and alternate the device back and forth from between the two modes. Similarly, primary probe 16 also includes a probe intensity dial 44 for adjusting the strength of the current applied by the electrodes. The operator may increase or decrease the amperage of the current sent through the electrodes and through the treated region of the patient by adjusting this knob using a thumb or finger.

Secondary probe 18 also has a probe body 48 with a protruding electrode 50. Electrode 50 has a rounded tip 52 and is designed similar to the electrode and tip of the primary probe. Secondary probe is connected by wire 49 to the electrotherapy device case 34 by which current is supplied to the electrode 50. Those skilled in the art will appreciate that the bodies 38 and 48 of probes 16 and 18 respectively as depicted in FIG. 2 are representative and may be comprised of any number of shapes and designs.

In this embodiment, adjusting knob 44 and toggle switch 42 are shown placed on the same probe. However, those skilled in the art will appreciate that these controls may be located on different probes. Similarly, the controls may be redundantly placed on both probes. Knob 44 and switch 42 may also be comprised of any suitable type of switching or adjustment mechanism. The placement of these two controls on one of the probes allows the operator to engage the controls without taking his or her hands off the probes or altering the placement of the electrodes on the afflicted region of the patient.

Electrotherapeutic device 14 also preferably includes a variety of controls on the casing 34. Control 60, volume, adjusts the volume of the sound emitted by speaker 36 and corresponding light readout. Control 62, tone, adjusts the frequency of the audible tone to communicate the level of conductivity between the probes. Control 63, intensity, controls the amount of carrier voltage. Control 64, sensitivity, adjusts the resistance range that will be shown on the visual indicator and heard on the audio indicator. Control 65, tone cut-off, adjusts the response level at which an auditory signal will be heard. Control 66, carrier, controls the frequency of the D.C. pulses. Control 67, polarity button: changes polarity of the pulse created between the primary and the secondary probes.

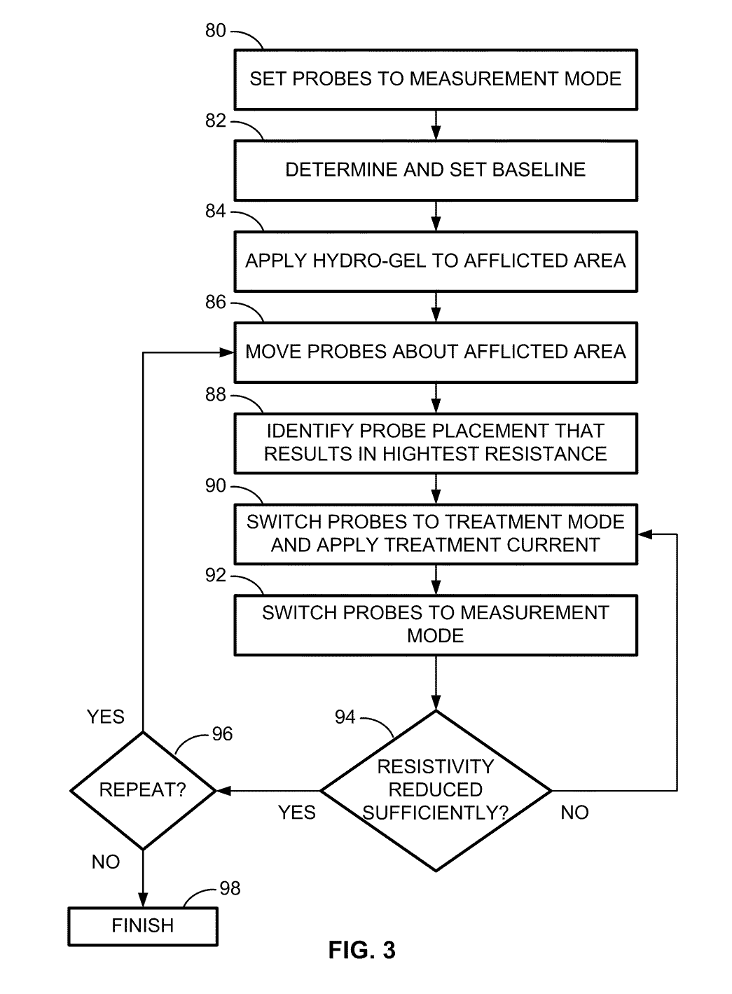

FIG. 3 shows a flowchart of the preferred method used with a preferred embodiment of the electrotherapy system of the invention. First, the electrotherapy device set up 80 in which it is turned on, set to the measuring mode and the voltage and amperage of the device is turned down. This may be accomplished by using the dial on the probe or the dials on the device case.

In the second step 82, a baseline resistivity/conductance is determined for the patient. This is typically accomplished by identifying an area on the patient similar to the region to be treated but lacking any malady for a baseline measurement. For example, if the patient's right leg is to be treated, as shown in FIG. 1, then the patient's left leg may be used to establish a baseline. The area chosen will then have a small amount of hydro-gel or other material to facilitate conductance of current into the patient is applied and the probes of the invention will be placed on the region. A measuring current is used to identify typical resistivity of the patient. The value assigned to this resistance level is assigned as the baseline level. The numerical value shown in the display is written down and recorded as the baseline if the display is utilized for displaying levels of resistivity. The volume and tone controls may be adjusted to the operator's preference.

In the next step 84, hydro-gel is applied to the area to be treated. Typically, an area experiencing pain, bruising or other malady has no precise, readily identifiable points ideal for application of electrotherapy. Therefore, the hydro-gel is applied to the general region about which the affliction to be treated is present.

In the following step 86, the operator places the electrodes protruding from the probes into direct contact with the patient's skin in the region to be treated. The ends of the probes are moved about the region relatively slowly as the electrotherapy device measures the resistance through the patient as it varies depending upon placement of the probes.

In step 88, the operator identifies the placement of the probes that experiences the greatest electrical resistance as measured by the electrotherapy device. As the probes are moved about the region to be treated, the resistance experienced by the measuring current varies resulting in the sound emitted varying. High resistance results in lower frequency sound. Therefore, the operator moves the probes about the patient's afflicted region until he or she identifies which positions of the probes causes the lowest tone to be emitted by the device. The highest resistance encountered may be recorded. By listening to the variance in tone of the sound emitted by the device, the operator can accurately identify preferable positionings of the probes without looking away from the region being treated. Those skilled in the art will appreciate that this is advantageous.

Once these positions are determined, the operator toggles the treatment switch so that the device applies a pulsed D.C. current between the probes in step 90. This sends a pulsed treatment current into the region of the patient between the probes. This is typically done for about 30 seconds. The length of time that the treating current is applied may vary according to how great the resistance measured was as compared to the baseline resistance of the patient.

After the initial treatment, the device is switched back to the measuring mode by toggling the switch on the primary probe in step 92. Typically, after treatment with the treatment current, the amount of resistance in the region being treated drops measurably. The new value of resistance in the treated region may be recorded.

In following step 94, if the measured resistance is still relatively high, then the process is repeated, going back to step 90 and reapplying the treatment current. The strength of the treatment current and the length of time the treatment current is applied will vary and is determined according to the best judgment of the operator of the device.

If the resistance has been substantially reduced and/or the pain has decreased sufficiently, then in step 96 the operator will determine whether the process should be repeated, and other placements of the probes exhibiting high resistance should be sought and treated. Again, this determination is primarily made according to the judgment of the operator.

If there is no longer any need at the time for further treatment, then the operator proceeds to step 98 and is finished. Otherwise, the operator returns to step 86 repeats the whole process. In this manner, the invention may be used to aid in the alleviation of a variety of ailments and maladies as described above and potentially others not yet identified, including ailments for which electrotherapy is currently contraindicated.

Those skilled in the art will appreciate that the method of the invention may be performed using separate measurement and treatment probes. However, this is generally not preferred because it increases the chances that the treatment current will not be applied at the precise location identified during the measuring of the resistance of the region being treated. It will also be appreciated that it is not necessary to place the controls directly on the probes or to use an audio tone to indicate to the operator the level of resistance experienced by the measuring current. However, a skilled artisan will recognize the advantages of utilizing these features of the invention in a synergistic fashion to enhance the efficiency and efficacy of the system and combined method and device of the invention.

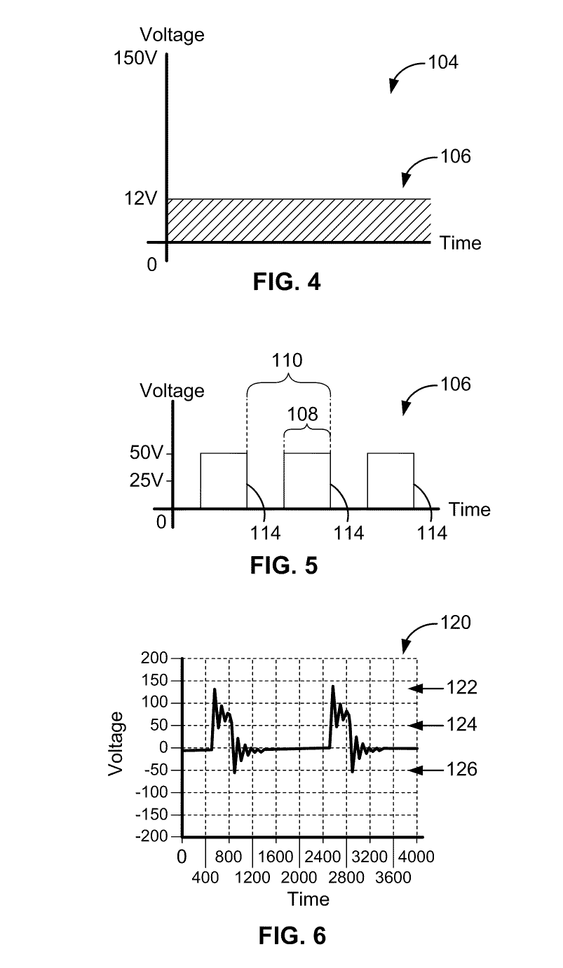

FIG. 4 shows a graph 104 of a typical applied voltage for measuring resistance 106. Preferably, the voltage 106 remains steady at approximately 12 volts. This creates a current passing from one electrode to the other through the body resistance. This is a strong enough current to accurately measure the resistance and conductance of the region being diagnosed without causing pain to the patient. Those skilled in the art will appreciate that 12 volts is only one of many acceptable levels for use with the invention and that a weaker or stronger current may be used. A voltage ranging between 7 and 12 is most preferably, however any convenient voltage that does not create significant discomfort is suitable. It is typically preferred to use a direct current and measure resistance when identifying the proper positioning of the electrodes rather than using an alternating current and measuring impedance. However, measuring in this manner, i.e. impedance of A.C. may be performed within the scope of the invention.

FIG. 5 shows a graph 109 of a typical treatment current 111. Treatment current 111 is a square wave created by a D.C. pulse. Preferably the current consists of a 420 (microsecond) 108 pulse burst 114 every 2.2 ms (milliseconds) 110 such that it operates at approximately 450 Hz. The nature of the pulsed direct current burst may vary greatly, and frequencies ranging from 100 to 1,000 Hz may be suitable. Generally, frequencies ranging from about 330 Hz to about 600 Hz are preferred. The synergy discovered from combining use of a measuring current and a treatment current is a salient feature of the invention, and modifications in the frequency of the pulses of the treatment current itself may vary considerably depending on the malady being treated and other factors.

Preferably, the strength of each burst is about 50 volts. The duration of the pulse, the frequency and the voltage may all be varied independently according to the judgment of the operator and the affliction being treated. This treatment wave is a preferred embodiment only and is not considered limiting other than to show that a square D.C. wave is preferred.

FIG. 6 shows and actual output waveform 120 of a preferred embodiment. D.C. pulsed burst wave 121 is shown having an approximate strength of about 50-80 volts. This may vary greatly, depending on a variety of factors. Those skilled in the art will appreciate that the actual pulsed current used therapeutically in the invention is only an approximation of a square wave, and does not have to be a perfect square wave in order to render treatment. This pulse begins with an initial burst 122 substantially greater than the average value of the burst and ends with ringing at the negative dip in voltage 126.

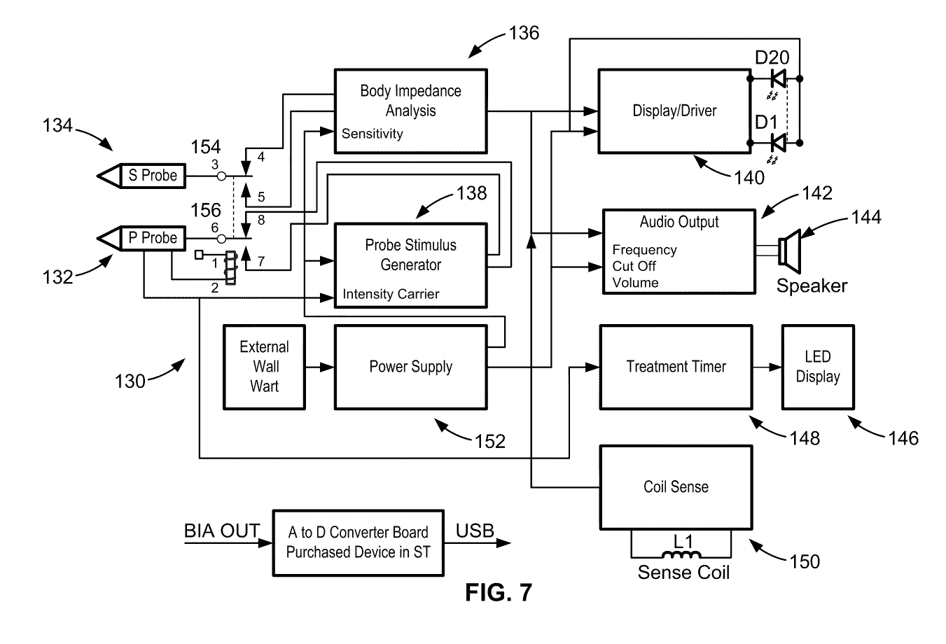

FIG. 7 shows a schematic diagram of device 130. Primary probe 132 and secondary probe 134 are connected to device 130 through input port 154 and input port 156. Primary probe 132 further is wired to probe stimulus generator 138. Both primary probe 132 and secondary probe 134 are connected to body impedance analyzer 136. Body impedance analyzer 136 and probe stimulus generator 138 provide the current necessary to measure resistance and the pulsed current necessary to treat a patient. Display driver 140 includes a light readout typically comprised of a long row of lights. Display driver 140 indicates the level of resistance or conductance passing between the electrodes and through the patient. Audio output 142 and speaker 144 are another means to determine the level of resistance or conductance passing between the electrodes and through the patients. Audio output 142 and speaker 144 are beneficial because they produce an audio signal that allows the operator to focus on the patient rather than device 130 during treatment. Treatment timer 148 sends the quantified value for the level of resistance or conductance within the current during diagnosis to LED display 146. Coil sense 150 may optionally be included for analysis of local magnetic fields about the region of the patient to be treated. Generally coil sense 150 is not essential to the operation of the invention. Power supply 152 generates all the power necessary for successful operation of device 130.

The invention is intended primarily for patients who experience the ailments described above or the like. However, the invention may also be used with animals or tissues before during and after transplantation. It may also be therapeutic to apply the invention to other organic material including plant life.

Whereas, the present invention has been described in relation to the drawings attached hereto, it should be understood that other and further modifications, apart from those shown or suggested herein, may be made within the spirit and scope of this invention. Descriptions of the embodiments shown in the drawings should not be construed as limiting or defining the ordinary and plain meanings of the terms of the claims unless such is explicitly indicated.

What is claimed is:

1. An electrotherapy apparatus comprising:
a primary probe having an electrode;
a secondary probe having an electrode;
electronic circuitry for generating a measuring current between the primary probe and the secondary probe and through a material placed between the probes;
electronic circuitry for measuring a resistivity of the material placed between the probes when the measuring current is generated;
at least one output for indicating the resistivity of the material;
electronic circuitry for generating a treatment current between the primary probe and the secondary probe and through the material placed between the probes, wherein the treatment current is a pulsed direct current.

2. The electrotherapy apparatus of claim 1 wherein the primary probe includes a treatment switch for changing a current generated between the primary probe and the secondary probe from the measuring current to the treatment current.

3. The electrotherapy apparatus of claim 1 wherein the primary probe includes a dial for increasing and decreasing the current generated between the primary probe and the secondary probe.

4. The electrotherapy apparatus of claim 2 wherein the at least one output comprises a visual readout and an audio signal and wherein the primary probe includes a dial for increasing and decreasing the current generated between the primary probe and the secondary probe.

5. The electrotherapy apparatus of claim 4 wherein the measuring current is a direct current having an open circuit potential between 5 and 25 volts.

6. The electrotherapy apparatus of claim 4 wherein the treatment current comprises a square wave having bursts of about 50 volts between 100 and 1,000 hertz.

7. A method for performing electrotherapy comprising:
measuring the electrical resistance between two electrodes placed at multiple locations within a region of the patient to be treated;
identifying the placement of the two electrodes which exhibits the highest resistance within the region of the patient to be treated;
applying a pulsed direct current through electrodes at the identified placement of the two electrodes which exhibits the highest resistance;
measuring again the electrical resistance between two electrodes placed at the identified placement;
responsive to the measured resistance between the electrodes not being substantially equivalent to the determined baseline of the patient, reapplying the pulsed direct current.

8. The method of claim 7 wherein the resistance between the electrodes is measured using a direct current having an open circuit potential between 5 and 25 volts.

9. The method of claim 7 wherein the pulsed direct current comprises a square wave having bursts of about 50 volts and a frequency between 100 and 1000 hertz.

10. The method of claim 7 wherein the pulsed direct current is applied using the same electrodes used to measure resistance.

11. The method of claim 7 wherein the measuring of resistance is performed using a primary probe and a secondary probe each having an electrode and being capable of applying both a steady direct current having an open circuit potential between 5 and 25 volts and a pulsed direct current comprising a square wave having bursts of about 50 volts between 350 and 550 hertz.

12. The method of claim 7 wherein the measuring of resistance is performed using an audio signal to indicate resistance within the material.

13. The method of claim 7 further comprising first measuring a baseline level of electrical resistance of a patient in an area of the patient unaffected by pain.

14. The method of claim 13 wherein the resistance between the electrodes is measured using a direct current having an open circuit potential between 5 and 25 volts.

15. The method of claim 7 further comprising applying a hydro-gel to facilitate conductance of current into the patient.

16. The method of claim 13 wherein the pulsed direct current is applied using the same electrodes used to measure resistance.

17. The method of claim 13 wherein the measuring of resistance is performed using a primary probe and a secondary probe each having an electrode and being capable of applying both a steady direct current having an open circuit potential between 5 and 25 volts and a pulsed direct current comprising a square wave having bursts of about 50 volts between 350 and 550 hertz.

18. The method of claim 13 wherein the measuring of resistance is performed using an audio signal to indicate resistance within the material.

19. A method for performing electrotherapy comprising:
measuring a baseline level of electrical resistance of a patient;
measuring the electrical resistance between two electrodes placed at multiple locations within a region of the patient to be treated, wherein the measuring is performed using a direct current of between 5 and 15 volts;
identifying the placement of the two electrodes which exhibits the highest resistance within the region of the patient to be treated, wherein the identifying accomplished by listening to an audio signal as the probes are moved about the region of the patient to be treated;
applying a pulsed direct current through electrodes at the identified placement of the two electrodes which exhibits the highest resistance, wherein the pulsed direct current is between 40 and 150 volts at a frequency of between 300 and 550 hertz;
measuring again the electrical resistance between two electrodes placed at the identified placement;
if the measured resistance between the electrodes is not substantially equivalent to the determined baseline of the patient, the reapplying the pulsed direct current.

20. The method of claim 19 wherein the measuring is recorded using quantified values provided on a digital readout.
